(19) United States
(12) Patent Application Publication
(10) Pub. No.: US 2026/0115348 A1
(43) Pub. Date: Apr. 30, 2026

(54) ETHYLENE AIR SCRUBBER FOR CARGO CONTAINERS AND CARGO CONTAINERS INCLUDING THE SAME

(71) Applicant: Advanced Composite Structures, LLC, Charleston, SC (US)

(72) Inventor: Thomas R. Pherson, Daniel Island, SC (US)

(73) Assignee: Advanced Composite Structures, LLC, Charleston, SC (US)

(21) Appl. No.: 19/370,902

(22) Filed: Oct. 28, 2025

Related U.S. Application Data
(60) Provisional application No. 63/713,619, filed on Oct. 30, 2024.

(51) Int. Cl. A61L 9/14; B65D 88/74

(52) U.S. Cl. CPC A61L 9/145; B65D 88/74; A61L 2209/13; A61L 2209/21; B65D 2588/74

(57) ABSTRACT

An air scrubber includes a tray and a cover screen. The tray has walls that define a perimeter of the tray. The tray also includes tabs that extend upward from opposite walls of the tray with each tab configured to engage a sidewall of a thermal protection module to mount the tray thereto. The cover screen is received within the perimeter of the tray and includes a base and raised elements that extend upward from the base. Each raised element defines a cavity with the base that is configured to retain a reactive material therewithin. The cover screen is configured to retain the reactive material while allowing air to pass through the reactive material such that the reactive material reduces undesirable compounds in the air.

### CROSS-REFERENCE TO RELATED APPLICATION

This application claims benefit of, and priority to, U.S. Provisional Patent Application Ser. No. 63/713,619, filed Oct. 30, 2024, the entire contents of which are hereby incorporated by reference.

### BACKGROUND

### 1. Technical Field

The present disclosure relates to cargo containers and, more specifically, to ethylene air scrubbers for use in cargo containers.

### 2. Discussion of Related Art

The ripening process of fruit generally produces ethylene (C₂H₄) gas and can be a significant source of this gas. Ethylene acts as a ripening accelerator creating an exponential increase of this gas and thus the ripening process when confined to a small volume such as a cargo container.

A significant amount of high value fruit such as grapes and avocados are shipped by air cargo because of their rapid ripening profile. An air cargo container is generally a closed vessel that can allow ethylene to accumulate and accelerate the ripening process in an uncontrolled and often unpredictable manner leading to uneven ripening including over ripening of the fruit throughout the cargo container. This uneven ripening and/or over ripening of the fruit may result in loss of product or reduced customer satisfaction. It is estimated that up to 30% of all produce is lost or damaged during the transportation process. The loss of product can have a significant cost.

The rate of all chemical reactions including ripening is governed by the Arrhenius Equation k=Ae\hat{ }(−Ea/RT) where k is the reaction rate, A is Arrhenius Factor, Ea is activation energy, R is universal gas constant, and T is absolute temperature. Without getting into detail, the net result of this equation is that the rate of ripening in this case is exponentially proportional to the absolute temperature of the ambient air. As a result, fruit may be refrigerated or cooled to slow ripening during transport. However, fruit may also be damaged by being kept too cold or being frozen. Thus, it may be important to keep fruit within a desired temperature range during shipping to prevent damage from being too cold and over ripening from being too warm.

Air cargo is typically transported in a cargo container generally referred to as Unit Load Device (“ULD”), which is stowed in a cargo hold of an aircraft, which can either be below and/or above the deck, e.g., below the deck in a passenger aircraft or below and above the deck in a transport aircraft. The outer size and shape of ULDs vary depending upon the type of aircraft such that the outer dimensions of the ULDs are determined by the type of aircraft. Typically, and regardless of the shape or geometry of the container, one end or side of the ULD is open for loading and unloading cargo. Various door closures can be used for opening and closing the open ends of the ULDs. The unloaded weight of the ULD is significant as even a slight reduction in the unloaded weight of the ULD will result in substantial savings in the cost of fuel to transport the ULD over its life. In addition, a reduction in the unloaded weight of the ULD will allow for an increased weight capacity for cargo.

Transporting perishable air cargo, such as fruit, may require a ULD to be insulated and/or refrigerated. Some perishable air cargo may require an interior of a ULD to be maintained below a specific temperature or within a specific temperature range. A ULD may include insulation either in the walls or disposed on the inside of the ULD such that the interior of the ULD is insulated. During the transport of perishable cargo, a ULD may include active or passive cooling therein to maintain a temperature within a desired temperature range.

### SUMMARY

This disclosure relates generally to ethylene air scrubbers that work in conjunction with thermal protection modules to remove or absorb ethylene from air flowing through the thermal protection modules within a cargo container. The thermal protection modules may create passive air circulation within the cargo container such that the air passes through the ethylene air scrubbers. In some embodiments, the cargo container may include active air circulation.

In an aspect of the present disclosure, an air scrubber includes a tray and a cover screen. The tray has walls that define a perimeter of the tray. The tray also includes tabs that extend upward from opposite walls of the tray with each tab configured to engage a sidewall of a thermal protection module to mount the tray thereto. The cover screen is received within the perimeter of the tray and includes a base and raised elements that extend upward from the base. Each raised element defines a cavity with the base that is configured to retain a reactive material therewithin. The cover screen is configured to retain the reactive material while allowing air to pass through the reactive material such that the reactive material reduces undesirable compounds in the air.

In aspects, the air scrubber further includes a bottom screen that is disposed between the tray and the cover screen. The bottom screen may be configured to retain the reactive material from passing through the tray. The tray may include supports that extend between the walls. The supports may prevent the bottom screen from passing through the tray. The bottom screen and the cover screen may be secured to the supports by one or more fasteners that pass through the screens and about the mounts. The bottom screen and the cover screen may be secured to the supports by fasteners passing through the screens and into the mounts.

In some aspects, the air scrubber includes a reactive material. The reactive material may be potassium permanganate that is received within the cavities of the cover screen and configured to react with ethylene to slow ripening of fruit within a cargo container including the air scrubber.

In another aspect of the present disclosure, a thermal protection module includes a pair of end caps, a plurality of heat transfer elements, and a first air scrubber. Each end cap is disposed at an opposite end of the thermal protection module. Each end cap includes a hanger that is configured to mount the thermal protection module to a ceiling of a cargo container. Each heat transfer element of the plurality of heat transfer elements extends between the pair of end caps and defines a gap with an adjacent heat transfer element. Each heat transfer element is filled with the phase change material. The plurality of heat transfer elements are configured to cooperate with one another to create a passive convective flow through the gaps. A pair of heat transfer elements of the plurality of heat transfer elements that define the outermost sides of the thermal protection module defined side walls on opposite sides of the thermal protection module. The first air scrubber includes a tray and a cover screen. The tray has walls that define a perimeter of the tray. The tray also includes tabs that extend upward from opposite walls of the tray. Each tab is engaged with one of the side walls of the thermal protection module to mount the tray to the thermal protection module with the walls disposed beneath the plurality of heat transfer elements. The cover screen is received within the perimeter of the tray. The cover screen includes a base and raised elements that extend upward from the base. Each raised element defines a cavity with the base. Each cavity is configured to retain a reactive material within the cavity and to allow air to pass from the gaps through the reactive material such that the reactive material reduces undesirable compounds in the air.

In aspects, each tab of the tray includes a grip that is configured to engage the respective side wall to mount the tray to the thermal protection module. Each tab may be deflected away from the opposite tab by the respective side wall. Each tab may be self-biased towards the opposite tab. The grip may be wedge shaped with a notch positioned at a bottom of the grip. The side wall may include a shelf that extends outward along the bottom edge of the side wall with the notch engaging the shelf to mount the tray to the respective side wall.

In some aspects, a portion of each raised element of the cover screen is partially disposed within a respective gap. The thermal protection module may include a second air scrubber that is mounted to the side walls of the thermal protection module and is spaced apart from the first air scrubber along the length of the thermal protection module. The second air scrubber may include a tray and a cover screen. The tray may have walls that define a perimeter of the tray and include tabs extending upward from opposite walls of the tray. Each tab may be engaged with one of the side walls of the thermal protection module to mount the tray to the thermal protection module with the walls disposed beneath the plurality of heat transfer elements. The cover screen may be received within the perimeter of the tray and include a base and raised elements that extend upward from the base. Each raised element may define a cavity with the base that is configured to retain a reactive material there within. The cover screen may be configured to retain the reactive material such that air is allowed to pass from the gaps through the reactive material such that the reactive material reduces undesirable compounds in the air. The reactive material of the first air scrubber may be the same or different from the reactive material of the second air scrubber.

In another aspect of the present disclosure, a method includes mounting air scrubbers to a thermal protection module that is secured within a cargo container. The method includes loading the cargo container with perishable cargo and transporting the cargo container with the perishable cargo. The air scrubbers receiving air from the thermal protection modules during transport such that the air reacts with the reactive material disposed within the air scrubbers to reduce an undesirable compound in the air. The perishable cargo produces the undesirable compound during transport.

In aspects, loading the cargo container with perishable cargo includes loading the cargo container with fruit or vegetables. The fruit or vegetables may produce ethylene during transport and the reactive material may remove ethylene from the air. The method may include creating a passive convective airflow within the cargo container with the thermal protection modules during transport of the cargo container such that air flows from a respective thermal protection module directly into one or more of the air scrubbers.

In some aspects, the method includes unloading the perishable cargo after transporting the cargo container, loading new perishable cargo into the cargo container after unloading the original perishable cargo, and transporting the new perishable cargo to another location. The air scrubbers may receive air from the thermal protection modules during transport such that the air reacts with the reactive material disposed within the air scrubbers to reduce the undesirable compound in the air. The new perishable cargo may produce the undesirable compound during transport.

In certain aspects the method includes replacing the reactive material within the air scrubbers after unloading the original perishable cargo and before loading the new perishable cargo.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are not necessarily drawn to scale, which are incorporated in and constitute a part of this specification, wherein:

FIG. 1 is a perspective view of a cargo container according to an embodiment of the present disclosure;

FIG. 2 is a cutaway view of a top portion of the cargo container of FIG. 1 showing an interior of the cargo container including thermal protection modules and air scrubbers secured to a ceiling of the cargo container in accordance with embodiments of the present disclosure;

FIG. 3 is an enlarged front view of one of the thermal protection modules of FIG. 2;

FIG. 4 is a top perspective view of one of the thermal protection modules of FIG. 2 with six air scrubbers mounted thereto;

FIG. 5 is a top perspective view of an air scrubber provided in accordance with embodiments of the present disclosure;

FIG. 6 is a top perspective view, with parts separated, of the air scrubber of FIG. 5;

FIG. 7 is a bottom perspective view of a cover screen of the air scrubber of FIG. 5;

FIG. 8 is a schematic cross-sectional view of the thermal protection module and air scrubber taken along section line 8-8 of FIG. 4; and

FIG. 9 is a flow chart of a method of removing or regulating air within a cargo container provided in accordance with embodiments of the present disclosure.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships, or the like, unless otherwise stated, any one or more if not all of these may be absolute or approximate to account for acceptable variations that may occur, such as those due to manufacturing or engineering tolerances or the like.

As used in the description and the appended claims, the phrases “unit load device” (ULD) or “air cargo container,” is defined as cargo containers used to load luggage, freight including perishable cargo, mail, and the like on aircraft including wide-body aircraft and narrow-body aircraft. While the cargo containers described herein are directed to ULDs or cargo containers for use with aircraft, it is contemplated that cargo containers including the disclosed thermal protection modules may be used in other transportation vehicles such as trucks, trailers, ships, or trains such that the described use with aircraft should not be seen as limiting. In addition, while the thermal protection modules described herein are described for use with cargo containers, it is contemplated that the thermal protection modules may be used in any enclosure to regulate a temperature therewithin. Further, the thermal protection modules detailed herein may be used for transport containers of varying sizes. For example, the thermal protection modules detailed herein could be used for transportation of perishable cargo. Perishable cargo may include food (e.g., fruits or vegetables) or pharmaceuticals.

The temperature of cargo within a cargo container designed with thermal insulation properties in mind may extend how long cargo is able to maintain a desired internal temperature. The desired internal temperature may be above or below an ambient temperature. Specifically, above or below the ambient temperature while an aircraft idles on the ground waiting to take off, during flight, and during loading or unloading of the aircraft.

When a cargo container is exposed to an ambient environment, other factors may increase a temperature differential between an interior of the cargo container and the ambient environment. For example, the cargo container may be exposed to the sun which may increase a temperature within the interior of the cargo container.

A thermally insulated cargo container may be loaded with materials in insulative containers. When such a thermally insulated cargo container is exposed to an ambient environment, the air in the interior of the cargo container may quickly increase in temperature as the specific heat of air is low. The quick increase in temperature of air within the cargo container may then increase a temperature of cargo within the cargo container such that the cargo is damaged or perishes. For example, as the temperature increases, the production of ethylene may increase which may cause runaway ripening of fruit or vegetables.

This disclosure is directed to ethylene air scrubbers for cargo containers which remove or absorb ethylene from air passing therethrough. The ethylene air scrubbers disclosed herein may be used with passive air flow or active air flow.

Referring now to FIG. 1, an example air cargo container or ULD is provided in accordance with the present disclosure and is referred to generally as container 100. As shown, the container 100 is a ULD for use below a deck of an aircraft. The container 100 may be designed to load luggage, freight, or mail in an aircraft. In this regard, the cargo container 100 may have other shapes for a position within a given aircraft or for a type of a given aircraft. The container 100 may include a frame presenting a generally rectangular shape with an offset designed to more closely follow the outline of the aircraft. The container 100 may further include a cargo opening defined by a portion of the frame. The frame may be formed from any substantially rigid material, such as aluminum, steel, composites, temperature resistant plastics, other metals, or other non-metals.

The frame may support a plurality of panels 104 forming the walls and the roof of the container 100. The container 100 may include a floor or a base 108 that allows the container 100 to be lifted by lifting equipment such as a forklift. In some embodiments, the panels 104 may be constructed together such that a separate frame, e.g., frame, may be eliminated. The panels 104 may be lightweight, thermal insulating, and/or have high strength characteristics. The cargo opening may be substantially sealed, and selectively closed, by a door 106. The door 106 may be a rigid door or may be a flexible door or curtain. When the door 106 is a rigid door, the door 106 may have similar construction to any of the panels 104. Alternatively, the door 106 may be insulated in another manner allowing the door 106 to be flexible. In addition, the frame, the panels 104, and/or the door 106 may be fire resistant.

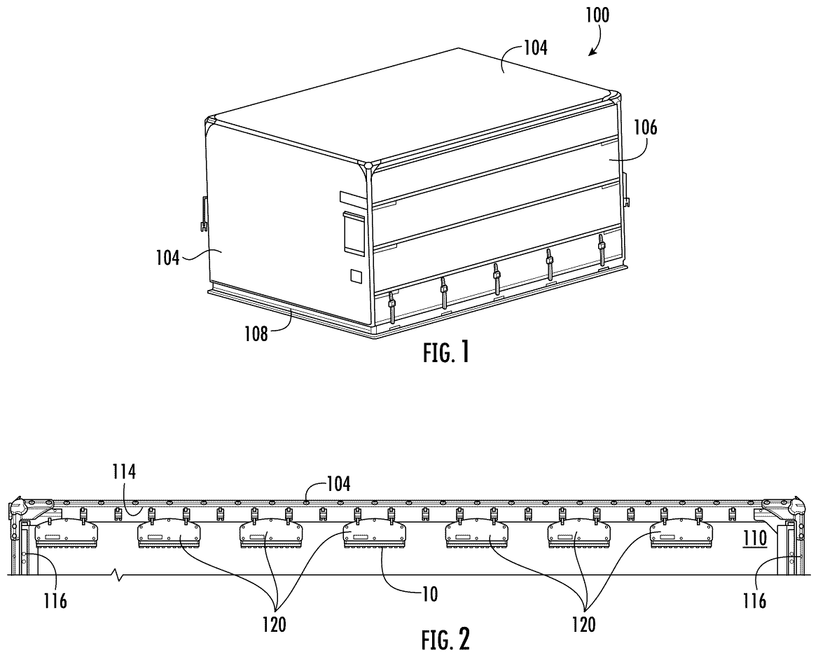

With additional reference to FIGS. 2 and 3, thermal protection modules 120 are secured to an interior of a cargo container 100. As shown, the thermal protection modules 120 may be secured to a ceiling 114 and/or walls 116 within a cavity 110. The thermal protection modules 120 may be secured to the ceiling 114 and/or walls 116 with the hangers 129. The hangers 129 are secured to the end caps of the thermal protection modules 120 and to mounting points 115 on the ceiling 114 of the cargo container 100. The thermal protection modules 120 may be removed from the cargo container 100. Each thermal protection module 120 may include multiple hangers 129 secured to each end cap of the thermal protection module 120. In certain embodiments, the hangers 129 may be secured directly to the ceiling 114 of the cargo container 100. The thermal protection modules 120 include sidewalls 126 that extend between the end caps and on opposite sides of the thermal protection modules 120. Suitable thermal protection modules are disclosed in U.S. patent application Ser. No. 18/887,481, the entire contents of which are hereby incorporated by reference.

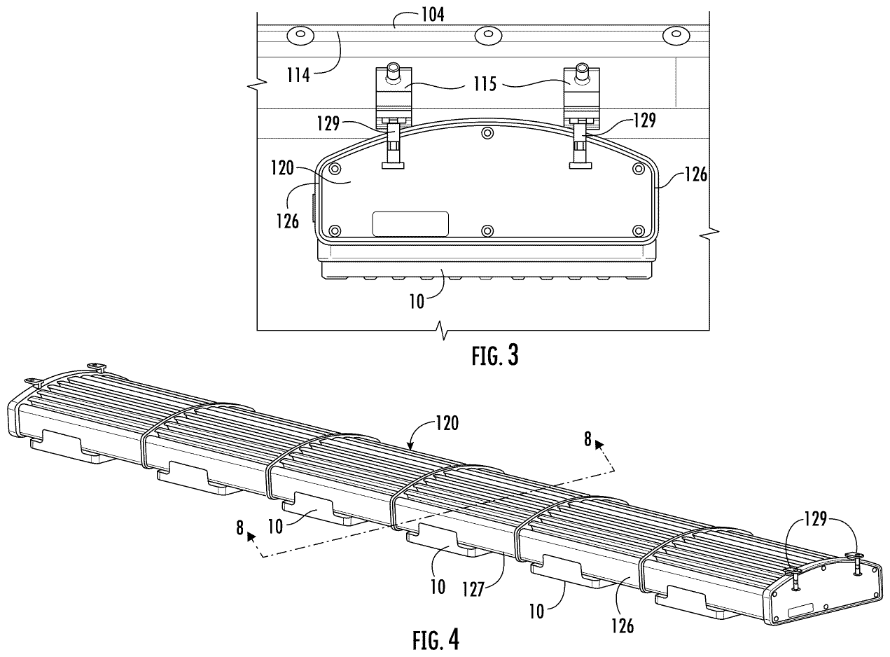

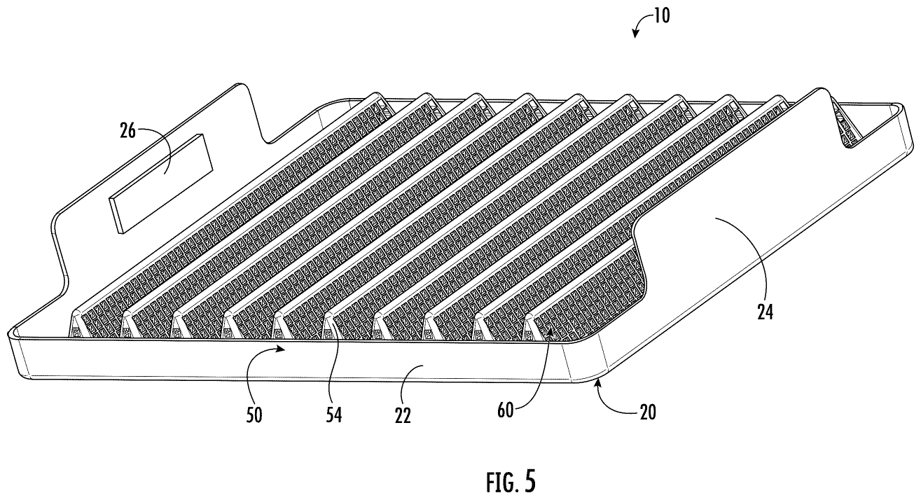

Referring now to FIG. 4, each thermal protection module 120 includes one or more air scrubbers 10 provided in accordance with the present disclosure. As shown, the thermal protection module 120 includes six air scrubbers 10 mounted to the sidewalls 126 of the thermal protection module 120 such that a tray 20 of the air scrubber 10 is positioned or slung beneath the thermal protection module 120. The air scrubbers 10 are configured to scrub air passing therethrough caused by passive convection currents produced by the thermal protection modules 120. The air scrubbers 10 may include materials that catalyze or absorb specific compounds in the air to reduce or eliminate a concentration of the compounds in the air. For example, the air scrubbers 10 may include materials that absorb ethylene (C₂H₄) from air passing therethrough. In some embodiments, the air scrubbers 10 may utilize active airflow to reduce or eliminate a concentration of the compounds in the air.

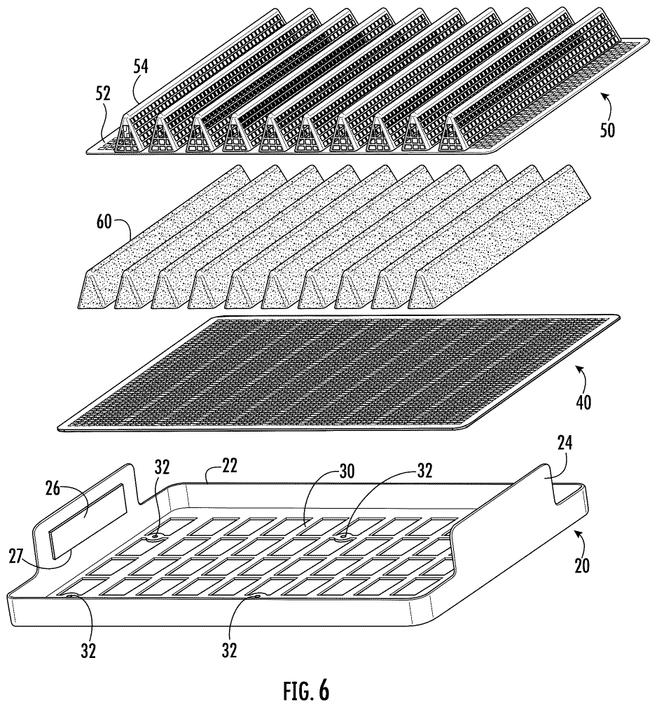

With reference to FIGS. 5 and 6, one of the air scrubbers 10 is described in accordance with the present disclosure. The air scrubber 10 includes a tray 20, a bottom screen 40, and a cover screen 50. The tray 20 includes walls 22 that define a perimeter of the tray 20. The walls 22 include tabs 24 that extend upward from the walls 22 on opposite sides of the tray 20. Each tab 24 may include a grip 26 that secures to an internal surface of the tab 24 such that the grips 26 oppose one another. The grips 26 may be additional material that is secured to the surface of the tab 24 such that the grip 26 extends from the internal surface. The grips 26 engage the sidewalls 126 of the thermal protection module 120 to mount the tray 20 to the thermal protection module 120. The grips 26 may be monolithically formed with the tab 24 or may be secured to the tab 24 by bonding, adhering, welding, or with one or more fasteners that pass therethrough. The grips 26 may be a material that is configured to frictionally engage the sidewalls 126 of the thermal protection module 120. The grips 26 may be formed of a material that absorbs or dampens vibration. The grips 26 may be formed of a thermally insulative material or may be formed of a material to enhance thermal conduction therethrough. In some embodiments, the grips 26 may be formed of rubber, a thermoplastic, a thermoset, or aluminum. The grips 26 may be formed of an attachment material that is configured to releasably engage the sidewalls 126 of the thermal protection module 120. In some embodiments, the grips 26 are two-part fasteners with a first part secured to the tabs 24 of the tray 20 and the second part secured to the sidewalls 126 of the thermal module 120. The two-part fasteners may be a hook and loop fastener or a mushroom hook and loop fastener that allows for secure placement and removal of the trays 20 from the sidewalls 126 of the thermal protection module 120.

The tray 20 includes a support 30 that extends between the walls 22 to provide support for the bottom screen 40 and the cover screen 50. The support 30 defines a substantially open structure to allow air to pass through the tray 20 while supporting the bottom screen 40 and the cover screen 50 as detailed below. The support 30 may include linear or arcuate structures that extend between the walls 22. The support 30 may form a linear grid pattern. The support 30 may include one or more mounts 32 that allow the bottom screen 40 or the cover screen 50 to secure to the tray 20. The support 30 may be monolithically formed with the tray 20 or the support 30 may be secured to the tray 20 by welding, adhering, bonding, or with one or more fasteners. In certain embodiments, the walls 22 may include a lip (not shown) that extends inward to retain the support 30 within the tray 20. The support 30 may be formed of the same or similar material to the tray 20. In some embodiments, the support 30 may be formed of a metal or metal alloy. In certain embodiments, the tray 20 is formed of an insulative material such as a thermoplastic or a thermoset such that the tray 20 is thermally insulated from the thermal protection module 120. In particular embodiments, the tray 20 and/or the support 30 may be formed of a thermally conductive material such that the tray 20 may conduct thermal energy from the thermal protection module 120 to extend a cooling or warming process from the thermal protection module 120. In such embodiments, the tray 20 and/or the support 30 may be formed of aluminum or other highly conductive material.

The bottom screen 40 is sized and dimensioned to fit within the tray 20 such that the bottom screen 40 substantially fills the bottom of the tray 20. The bottom screen 40 lays on the support 30 such that the bottom screen 40 is received within the tray 20. The bottom screen 40 supports a reactive material 60 within the tray 20. The bottom screen 40 may be in the form of a mesh screen that supports the reactive material 60 and allows air to freely pass through the bottom screen 40. In some embodiments, the bottom screen 40 lays directly on lips of the tray 20 such that the tray 20 may be provided without the support 30. In certain embodiments, one or more fasteners pass through the bottom screen 40 to secure the bottom screen 40 to the support 30. In certain embodiments, a fastener passes through the bottom screen 40 and into the mount 32 of the support to secure the bottom screen 40 to the support 30. In some embodiments, the fastener may be a ratcheting tie that passes through the bottom screen 40, about the support 30, and back through the bottom screen 40.

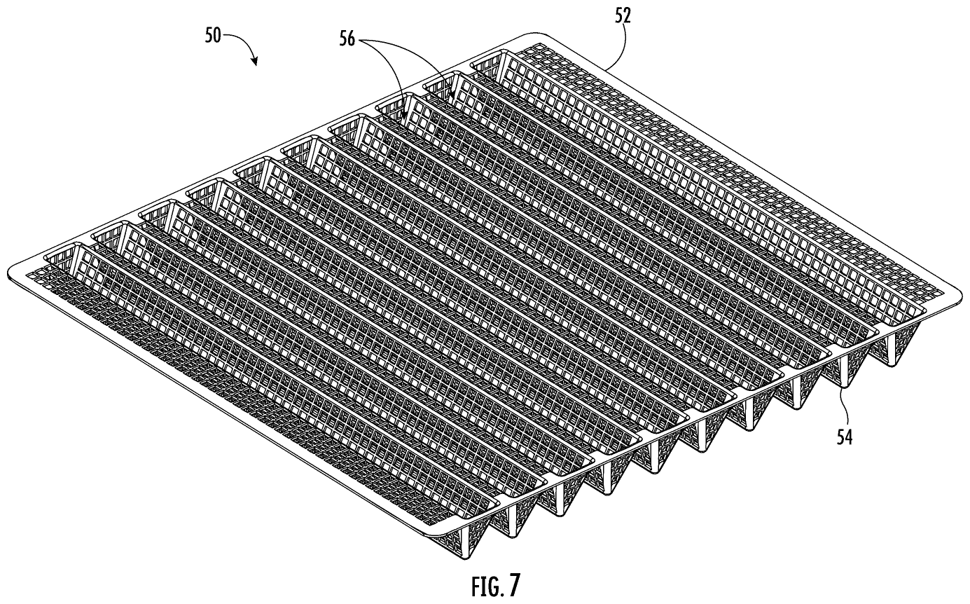

With additional reference to FIG. 7, the cover screen 50 is positioned in the tray 20 over the bottom screen 40 such that the reactive material 60 is retained between the bottom screen 40 and the cover screen 50. Similar to the bottom screen 40, the cover screen 50 may be in the form of a mesh that retains the reactive material 60 while allowing air to pass through the cover screen 50 or to selectively bypass the cover screen 50. The geometry of the cover screen 50 may minimize a pressure drop at low flow rates, e.g., passive convective flow rates. The cover screen 50 includes a base 52 and may include one or more raised features 54 that extend above the base 52 to each define a cavity 56 between the cover screen 50 and the base 52. The cavities 56 are each sized and dimensioned to receive the reactive material 60 therein. As shown, the raised features 54 are in the form of ridges that form triangular prisms with the base 52. The raised features 54 may increase a surface area on the top surface of the cover screen 50 to allow for increased surface contact with the reactive material 60. The peaks of the raised features 54 may be above the walls 22 of the tray 20. The ends of the raised features 54 may be closed with end plates such that the reactive material 60 is retained within the cavities 56. In certain embodiments, the peaks of the raised features 54 are below the walls 22 and the ends of the raised features 54 are open with the walls 22 retaining the reactive material 60 within the cavities 54. Other shapes are contemplated for the raised features 54 including semi-cylinders, cones, frustoconical features, or other prismatic structures. The cover screen 50 may be secured to the bottom screen 40 or the support 30 by one or more fasteners in a manner similar to the bottom screen 40. The bottom screen 40 and the cover screen 50 may be considered a screen assembly. In some embodiments, only the cover screen 50 may be considered the screen assembly.

The reactive material 60 may be in the form of free pellets or pellets within satchels or other packaging that allows air to pass through. The reactive material 60 may come preformed to fit within the cavities 56. The reactive material 60 is configured to absorb or react with compounds to remove the compounds from air passing through the reactive material 60. The reactive material 60 may be configured to remove ethylene (C₂H₄) from air passing through the reactive material 60. For example, the reactive material 60 may be potassium permanganate (KMnO₄) such that the ethylene is absorbed within the following reaction:

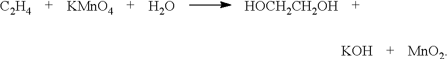

From the reaction above it is seen that using potassium permanganate to remove ethylene may also remove some moisture from the air. In some embodiments, the reactive material 60 may be pure potassium permanganate crystals or beads. In certain embodiments, the reactive material 60 may be potassium permanganate crystals or beads mixed with alumina. The potassium permanganate may be provided in disposable or refillable containers having an open volume greater than 30 percent, 40 percent, or 50 percent to allow airflow therethrough and greater surface contact with the air stream to the beads of reactive material 60. The beads may be available in diameters in a range of 2 millimeters to 6 millimeters with a concentration of potassium permanganate in a range of 6 percent to 15 percent. In some embodiments, the reactive material 60 may be selected to remove moisture, carbon dioxide (CO₂), hydrogen sulfide (H₂S), or other gases from the air.

The bottom screen 40 and the cover screen 50 are formed of a material that is non-reactive to the reactive material 60 or the compound being removed or reduced from the air. In some embodiments, the bottom screen 40 and the cover screen 50 are formed of the same material. In certain embodiments, the bottom screen 40 and the cover screen 50 are formed of different materials. The bottom screen 40 or the cover screen 50 may be formed of a thermoplastic, a thermoset, a metal such as aluminum or steel, or another suitable material.

The configuration of the air scrubber 10 may allow for a high flow of air to pass therethrough while exposing a large surface area of the reactive material 60 to the air flow. The configuration of the air scrubber 10 may have a low pressure drop in the air passing through making the configuration suitable for passive convection currents generated by the thermal protection modules 120.

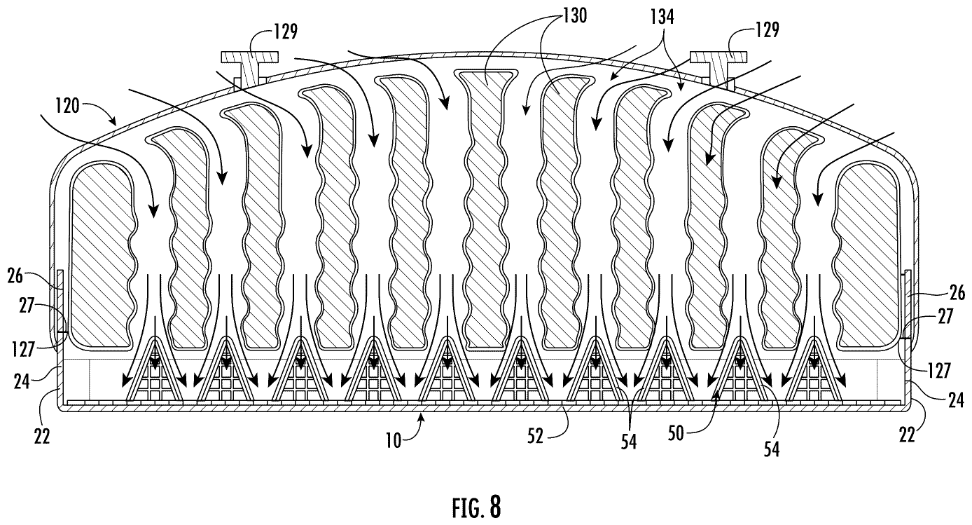

Referring now to FIG. 8, when the air scrubber 10 is mounted to the thermal protection module 120, air passing through the thermal protection module 120 flows through gaps 134 defined between adjacent elements 130 of the thermal protection module 120. As the air flows through the gaps 134, the air is cooled such that the cooler, denser air flows out the bottom of the thermal protection module 120. The peaks of the raised elements 54 may be disposed just within the gaps 34 such that as the air exits the gaps 34, it is directed into and along the sides of the raised elements 54 to contact the reactive material 60 within the air scrubbers 10. As the air passes through the air scrubbers 10, undesirable compounds react with the reactive material 60 to remove or reduce an amount of the undesirable compounds within the air.

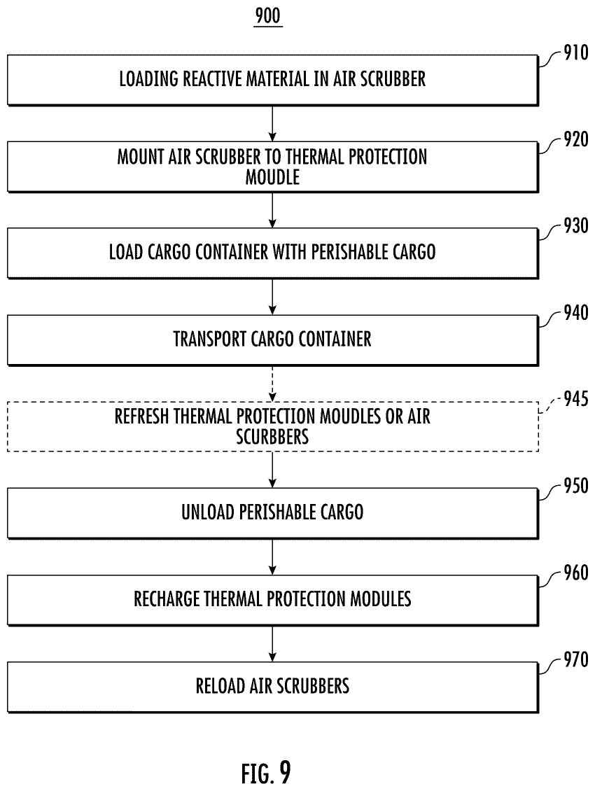

With reference to FIG. 9, a method of removing or regulating air within a cargo container referred to generally as method 900 is disclosed according to the present disclosure with reference to the cargo container 100, the thermal protection modules 120, and the air scrubbers 10 of FIGS. 1-8. The method 900 may be used to preserve sensitive cargo during transport. For example, perishable cargo such as fruit or vegetables is frequently transported in cargo containers. During transport, fruit or vegetables may emit ethylene gas as the fruit or vegetables ripens. In addition, exposure to ethylene hastens the ripening of fruit or vegetables such that if ethylene gas is not removed from the air, ripening fruit or vegetables will quickly spoil. The method 900 may be used to remove or reduce ethylene gas from the air to slow the ripening of fruit or vegetables within the cargo container. It is also noted that the ripening of fruit or vegetables may be hastened as the temperature increases.

The method 900 may include placing thermal protection modules 120 within a cargo container 100. The thermal protection modules 120 may be charged before being placed in the cargo container 100 or may be charged while in the cargo container 100. The method 900 may include loading a reactive material 60 into an air scrubber 10 (Step 910). The reactive material 60 may be loaded by positioning the reactive material 60 within cavities 56 of the cover screen 50. With the reactive material 60 within the cavities 56, the bottom screen 40 may be placed over the cavities 56 to close the cavities 56. With the cavities 56 closed, the bottom screen 40 and the cover screen 50 are placed on the tray 20. In some embodiments, the bottom screen 40 may be positioned in the tray 20 and the reactive material 60 may be placed on the bottom screen 40. With the reactive material 60 placed on the bottom screen 40, the cover screen 50 may be placed over the bottom screen 40 such that the reactive material 60 is received within the cavities 56 of the cover screen 54.

With the reactive material 60 loaded into the air scrubber 10, the air scrubber 10 is mounted to a thermal protection module 120 (Step 920). The air scrubber 10 may be mounted to the thermal protection module 120 when the thermal protection module 120 is secured to the ceiling 114 of a cargo container 100 or may be mounted to the thermal protection module 120 before the thermal protection module 120 is secured to the cargo container 100. The air scrubber 10 may be mounted to the thermal protection module 120 by engaging the sidewalls 126 of the thermal protection module 120 with the tabs 24 of the air scrubber 10. As the grips 26 engage the sidewalls 126, the tabs 24 may deflect or deform outwards to allow the grips 26 to slide over the sidewalls 126. The grips 26 may form a wedge with the top being thinner than the bottom such that when the bottom passes over the bottom of the sidewalls 126 a notch 27 of the grip 26 engages a shelf 127 of the sidewall 126 to retain the air scrubber 10 on the thermal protection module 120. The tabs 24 may be self-biased towards one another such that the grips 26 are urged towards one another to mount the tray 20 to the thermal protection module 120. In some embodiments, the tabs 24 may be formed such that the tabs 24 are angled slightly towards one another, e.g., 1 degree to 5 degrees, such that when the tabs 24 are positioned on the sidewalls 126, the tabs 24 are urged outward from the natural or resting position. The self-biasing or deformation of the tabs 24 may assist in mounting the air scrubber 10 to the thermal protection module 120. In some embodiments, the thermal protection modules may include removeable fasteners such as two-part hook and loop fasteners, snaps, or other similar fasteners to mount the air scrubbers 10 to the thermal protection modules 120. Each thermal protection module 120 may have a plurality of air scrubbers 10 mounted thereto. As shown, a single thermal protection module 120 has six air scrubbers 10 mounted thereto. In some embodiments, the number of air scrubbers for a cargo container 100 may be determined by a cargo, a temperature profile, a duration, a current ripened state of cargo, or combinations thereof. As such, a thermal protection module 120 may include between 0 and 12 air scrubbers 10 mounted thereto. In some embodiments, a thermal protection module 120 may include more than 12 air scrubbers mounted thereto.

With the air scrubbers 10 loaded and mounted to the thermal protection modules 120 and the thermal protection modules 120 secured to the ceiling 114 of the cargo container 100, the cargo container 100 may be loaded with perishable cargo (Step 930). The cargo container 100 may be loaded with cargo in a cold or cool room. It is contemplated that the thermal protection modules 120 and/or the air scrubbers 10 may be secured and mounted to the cargo container 100 in a cold or cool room or outside of a cold room or cool room. When the cargo container 100 is loaded with perishable cargo, the thermal protection modules 120, and the air scrubbers 10, the cargo container 100 is closed and transported to another location for unloading (Step 940). During transport, the thermal protection modules 120 may maintain a temperature within the cargo container 100 while creating passive convective flow within the cargo container 100 such that air flows through the thermal protection modules 120 and the air scrubbers 10. As air passes through the air scrubbers 10, the air scrubbers 10 remove or reduce undesirable compounds within the air. For example, the air scrubbers 10 may remove or reduce ethylene from the air passing therethrough. Removing an undesirable compound from the air during transport may prevent or delay the perishable cargo from perishing during transport. In some embodiments, the thermal protection modules 120 or the air scrubbers 10 may be refreshed during transport (Step 945). The thermal protection modules 120 or the air scrubbers 10 may be refreshed by opening the cargo container 100 and removing air scrubbers 10 or thermal protection modules 120 and replacing with new air scrubbers 10 or thermal protection modules 120. In some embodiments, the reactive material 60 may be unloaded and reloaded into select air scrubbers 10. However, while this is contemplated, it is activity that may only occur when there is an unplanned delay during transport beyond anticipated during planning. For example, a weather delay, a sudden shift in a temperature profile on the planned route, or other factor.

When the cargo container 100 arrives at its destination, the perishable cargo may be unloaded (Step 950). After unloading, the thermal protection modules 120 may be recharged (Step 960). Recharging of the thermal protection modules 120 may occur within the cargo container 100 or outside of the cargo container 100. In addition, the air scrubbers 10 may be reloaded with new reactive material 60 (Step 970). In some embodiments, the air scrubbers 10 may be used on multiple loads before being reloaded. In such embodiments, the life of the reactive material 60 may be analyzed after a cargo run to determine if the air scrubber 10 needs to be reloaded before a subsequent cargo run. In some embodiments, the reactive material 60 may be analyzed by a color change. For example, potassium permanganate beads may initially be purple and change to brown when converted to magnesium dioxide indicating that the reactive material 60 needs to be reloaded.

To reload the air scrubber 10, the air scrubber 10 is detached from the thermal protection module 120, the cover screen 54 is lifted and the reactive material 60 is removed from the cavities 56 of the cover screen 54. New reactive material 60 may then be loaded into the cavities 56 of the cover screen 54 for the next cargo run. In some embodiments, the reactive material 60 is removed after a cargo run and the air scrubber 10 is transported to another location before being reloaded. In certain embodiments, the reactive material 60 may be recharged such that it may be utilized for future cargo runs.

Although the method steps are described in a specific order, it should be understood that other steps may be performed in between described steps, described steps may be adjusted so that they occur at slightly different times, or the described steps may occur in any order unless otherwise specified.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. An air scrubber comprising:
a tray having walls, the walls defining a perimeter of the tray, the tray including tabs extending upward from opposite walls of the tray, each tab configured to engage a sidewall of a thermal protection module to mount the tray thereto; and
a cover screen received within the perimeter of the tray, the cover screen including a base and raised elements that extend upward from the base, each raised element defining a cavity with the base, each cavity configured to retain a reactive material therewithin and to allow air to pass through the reactive material such that the reactive material reduces undesirable compounds in the air.

2. The air scrubber according to claim 1, further comprising a bottom screen disposed between the tray and the cover screen, the bottom screen configured to retain the reactive material from passing through the tray.

3. The air scrubber according to claim 2, wherein the tray includes supports that extend between the walls, the supports preventing the bottom screen from passing through the tray.

4. The air scrubber according to claim 3, wherein the bottom screen and the cover screen are secured to the supports by one or more fasteners passing therethrough and about mounts of the supports.

5. The air scrubber according to claim 3, wherein the supports include mounts, the bottom screen and the cover screen secured to the supports by fasteners passing therethrough into the mounts.

6. The air scrubber according to claim 1, further comprising a reactive material including potassium permanganate, the reactive material received in the cavities of the cover screen and configured to react with ethylene to slow ripening of fruit within a cargo container including the air scrubber.

7. A thermal protection module comprising:
a pair of end caps, each end cap disposed at an opposite end of the thermal protection module;
a plurality of heat transfer elements, each heat transfer element of the plurality of heat transfer elements extending between the pair of end caps and defining a gap with an adjacent heat transfer element, each heat transfer element filled with a phase change material, the plurality of heat transfer elements configured to cooperate with one another to create a passive convective flow through the gaps, a pair of heat transfer elements of the plurality of heat transfer elements that define outermost sides of the thermal protection module defining sidewalls on opposite sides of the thermal protection module; and
a first air scrubber comprising:
a tray having walls, the walls defining a perimeter of the tray, the tray including tabs extending upward from opposite walls of the tray, each tab engaged with one of the sidewalls of the thermal protection module to mount the tray thereto with the walls disposed beneath the plurality of heat transfer elements; and
a cover screen received within the perimeter of the tray, the cover screen including a base and raised elements that extend upward from the base, each raised element defining a cavity with the base, each cavity configured to retain a reactive material therewithin and to allow air to pass from the gaps through the reactive material such that the reactive material reduces undesirable compounds in the air.

8. The thermal protection module according to claim 7, wherein each tab of the tray includes a grip, the grip configured to engage the respective sidewall to mount the tray thereto.

9. The thermal protection module according to claim 8, wherein each tab is deflected away from the opposite tab by the respective sidewall.

10. The thermal protection module according to claim 9, wherein each tab is self-biased towards the opposite tab.

11. The thermal protection module according to claim 8, wherein the grip is wedge shaped with a notch positioned at a bottom of the grip, the sidewall including a shelf extending outward along a bottom edge of the sidewall, the notch engaging the shelf to mount the tray to the respective sidewall.

12. The thermal protection module according to claim 7, wherein a portion of each raised element of the cover screen is partially disposed within a respective gap.

13. The thermal protection module according to claim 7, further comprising a second air scrubber mounted to the sidewalls of the thermal protection module and spaced apart from the first air scrubber, the second air scrubber comprising:
a tray having walls, the walls defining a perimeter of the tray, the tray including tabs extending upward from opposite walls of the tray, each tab engaged with one of the sidewalls of the thermal protection module to mount the tray thereto with the walls disposed beneath the plurality of heat transfer elements; and
a cover screen received within the perimeter of the tray, the cover screen including a base and raised elements that extend upward from the base, each raised element defining a cavity with the base, each cavity configured to retain a reactive material therewithin and to allow air pass from the gaps through the reactive material such that the reactive material reduces undesirable compounds in the air.

14. The thermal protection module according to claim 13, wherein the reactive material of the first air scrubber is different from the reactive material of the second air scrubber.

15. A cargo container comprising:
a first sidewall;
a second sidewall opposite the first sidewall;
a back wall extending between the first sidewall and the second sidewall;
an opening defined between the first sidewall and the second sidewall opposite the back wall;
a closure configured to selectively close the opening;
a ceiling disposed above and supported by the first sidewall, the second sidewall, and the back wall, the first sidewall, the second sidewall, the back wall, and the closure defining an interior of the cargo container; and
a first thermal protection module according to claim 7, each end cap including a hanger, the hangers securing the first thermal protection module to the ceiling of the cargo container.

16. A method of regulating air within a cargo container, the method comprising:
mounting air scrubbers to thermal protection modules secured within the cargo container;
loading the cargo container with perishable cargo; and
transporting the cargo container with the perishable cargo, the air scrubbers receiving air from the thermal protection modules during transport such that the air reacts with a reactive material disposed within the air scrubbers to reduce an undesirable compound in the air, the perishable cargo producing the undesirable compound during transport.

17. The method according to claim 16, wherein loading the cargo container with perishable cargo includes loading the cargo container with fruit or vegetables, the fruit or the vegetables producing ethylene during transport, the reactive material removing ethylene from the air.

18. The method according to claim 16, further comprising creating a passive convective air flow within the cargo container with the thermal protection modules during transport of the cargo container such that air flows from a respective thermal protection module directly into one or more of the air scrubbers.

19. The method according to claim 16, further comprising:
unloading the perishable cargo after transporting the cargo container;
loading new perishable cargo into the cargo container after unloading the original perishable cargo; and
transporting the new perishable cargo to another location, the air scrubbers receiving air from the thermal protection modules during transport such that the air reacts with the reactive material disposed within the air scrubbers to reduce the undesirable compound in the air, the new perishable cargo producing the undesirable compound during transport.

20. The method according to claim 19, further comprising:
replacing the reactive material within the air scrubbers after unloading the original perishable cargo and before loading the new perishable cargo; or
recharging the thermal protection modules after unloading the perishable cargo and before transporting the new perishable cargo.
